(19) United States
(12) Patent Application Publication
(10) Pub. No.: US 2019/0224187 A1
(43) Pub. Date: Jul. 25, 2019

(54) NEW USE OF ISOQUINOLINE DERIVATIVES FOR WOUND HEALING

(71) Applicant: ZIH YUAN TANG Biotechnology Co., Ltd, Taipei City (TW)

(72) Inventors: Ming-Jai SU, Taipei City (TW); Shoei-Sheng LEE, Taipei City (TW); Chao-Min HSU, New Taipei City (TW)

(73) Assignee: ZIH YUAN TANG Biotechnology Co., Ltd, Taipei City (TW)

(21) Appl. No.: 16/254,056

(22) Filed: Jan. 22, 2019

Related U.S. Application Data
(60) Provisional application No. 62/620,041, filed on Jan. 22, 2018.

(51) Int. Cl. A61K 31/472; A61P 17/02

(52) U.S. Cl. CPC A61K 31/472; A61P 17/02

(57) ABSTRACT

The present invention is related to a method for wound healing comprising administering to a subject in need thereof a pharmaceutical composition comprising a pharmaceutically acceptable carrier and a therapeutically effective amount of a compound having the general Formula I, preferably salsolinol.

### CROSS REFERENCE TO RELATED APPLICATIONS

This non-provisional application claims the benefit under 35 U.S.C. § 119(e) to U.S. Provisional Application No. 62/620,041, filed on Jan. 22, 2018, which is hereby expressly incorporated by reference into the present application.

### FIELD OF THE INVENTION

The present invention provides a method for wound healing. In particular, the present invention provides a new use of isoquinoline derivatives for wound healing.

### BACKGROUND OF THE INVENTION

Isoquinoline derivatives are a group of nitrogen-containing organic compounds existing in plants and animals in nature. Most of them have a complex ring structure with their nitrogen atom incorporated in the ring. Such isoquinoline derivatives, including salsolinol and reticuline, possess significant biological activities. Salsolinol is known to be used mainly for regulation of blood pressure, while reticuline is used mainly as an active ingredient for treating malaria, and also as a component in pain relievers.

It is disclosed in U.S. Pat. No. 9,707,222 that isoquinoline derivatives, i.e., salsolinol and reticuline, can activate AMP-dependent protein kinase (AMPK), and use in treatment of AMPK-dependent diseases. In particular, either salsolinol or reticuline has an efficacy in reducing blood glucose and the treatment of diabetes.

Further, U.S. Pat. No. 9,655,891 discloses a method for diabetic wound healing in a subject, which comprises applying to the diabetic wound in said subject a pharmaceutical composition comprising a pharmaceutically acceptable carrier and a therapeutically effective amount of an isoquinoline derivative, including salsolinol and reticuline. However, none of prior art references reports the effect of such isoquinoline derivatives, e.g., salsolinol, in wound healing.

### BREIF SUMMARY OF THE INVENTION

It is unexpectedly found in the present invention that certain isoquinoline derivatives, such as salsolinol, are effective in wound healing.

In one aspect, the present invention provides a method for wound healing comprising administering to a subject in need thereof a pharmaceutical composition comprising a pharmaceutically acceptable carrier and a therapeutically effective amount of a compound having the general Formula I:

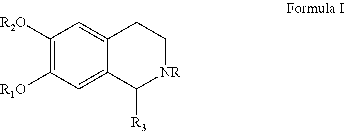

wherein R, R₁ and R₂ are each independently H, alkyl or acyl (RₐCO) group; R₃ is H, alkyl or substituted benzyl group; wherein Rₐ is H or alkyl group.

In one embodiment of the present invention, said substituted benzyl group has the following formula II:

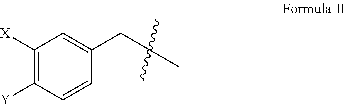

wherein X and Y are each independently H, OH, methoxy (OMe) or acyloxy (R_{b}CO—O—) group; wherein R_{b} is H or alkyl group.

In one example of the present invention, said compound having Formula I is salsolinol.

In another aspect, the present invention provides a use of a compound having the general Formula I of the present invention in manufacture of a medicament for wound healing.

Those and other aspects of the present invention may be further clarified by the following descriptions and drawings of preferred embodiments. Although there may be changes or modifications therein, they would not betray the spirit and scope of the novel ideas disclosed in the present invention.

### BRIEF DESCRIPTION OF THE DRAWINGS

The patent or application file contains at least one color drawing. Copies of this patent or patent application publication with color drawings will be provided by the USPTO upon request and payment of the necessary fee. The drawings presenting the preferred embodiments of the present invention are aimed at explaining the present invention. It should be understood that the present invention is not limited to the preferred embodiments shown. The data in the figures and examples are shown as mean±standard deviation (SD), determined by the paired t-test.

FIG. 1 shows the rates of the wound recovery in the animal treated with salsolinol at 0.01 mg/g at Day 1, Day 3, Day 5, Day 7, Day 10 and Day 14, as compared with the control group (treated with the vehicle) (*: p<0.01).

FIG. 2 shows the rates of the wound recovery in the animal treated with salsolinol at 0.03 mg/g at Day 1, Day 3, Day 5, Day 7, Day 10 and Day 14, as compared with the control group (treated with the vehicle) (**: p<0.05).

FIG. 3 provides the images of the wounds of one representative animal in each of the experiment and control groups at Day 1, Day 3, Day 5, Day 7, Day 10 and Day 14.

FIG. 4 provides the the wound recovery in the animal treated with salsolinol at 0.001 g/g at Day 1, Day 3, Day 5, Day 7, Day 10 and Day 14, as compared with the control group (treated with the vehicle).

FIG. 5 provides the the wound recovery in the guinea pigs treated with salsolinol at 0.01 mg/g at Day 1, Day 3, Day 5, Day 7, Day 10 and Day 14, as compared with the control group (treated with the vehicle) (**: p<0.05).

### DETAILED DESCRIPTION OF THE INVENTION

Unless defined otherwise, all technical and scientific terms used herein have the same meanings as commonly understood by one of skill in the art to which this invention belongs.

Unless clearly specified herein, meanings of the articles “a,” “an,” and “said” all include the plural form of “more than one.” Therefore, for example, when the term “a component” is used, it includes multiple said components and equivalents known to those of common knowledge in said field.

As used herein, the term “substituted” or “substitution” refers to where a functional group in a chemical compound is replaced by another group.

As used herein, the term “subject” refers to a human or a mammal, such as a patient, a companion animal (e.g., dog, cat, and the like), a farm animal (e.g., cow, sheep, pig, horse, and the like) or a laboratory animal (e.g., rat, mouse, rabbit, and the like).

The term “alkyl group” used herein refers to linear or branched monovalent hydrocarbons containing 1-20 carbon atoms, such as alkyl groups with 1-10 carbons, preferably alkyl groups with 1-6 carbons, more preferably alkyl groups with 1-3 carbons. Examples of alkyl groups include, but not limited to, methyl, ethyl, n-propyl, isopropyl, n-butyl, isobutyl, and t-butyl.

Accordingly, the invention, in one aspect, provides a method for wound healing. The method comprises administering to a subject in need thereof a pharmaceutical composition comprising a pharmaceutically acceptable carrier and a therapeutically effective amount of a compound having the general Formula I:

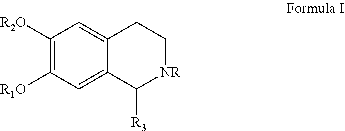

wherein R, R₁ and R₂ are each independently H, alkyl or acyl (RₐCO) group; R₃ is H, alkyl or substituted benzyl group; wherein Rₐ is H or alkyl group.

In a particular example of the present invention, said substituted benzyl group has the following Formula II:

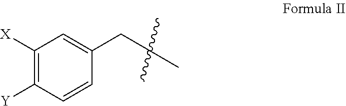

wherein X and Y are each independently H, OH, methoxy (OMe) or acyloxy (R_{b}CO—O—) group; wherein R_{b} is H or alkyl group.

An embodiment of the active compound of the present invention is the compound having the general Formula I, wherein R=R₁=R₂=H and R₃=Me (methyl), which compound is salsolinol having the following formula:

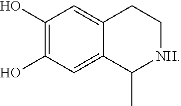

As shown in the examples of the present invention, the compound having Formula I of the present invention, e.g. salsolinol, has an effect of treating a wound.

It was unexpectedly found in the invention that the effective amount of salsolinol is 0.001 mg/g to 0.03 mg/g, preferably 0.01 mg/g.

According to the present invention, said compound having Formula I can be formulated into any forms of medications that are well known or commonly used in the pharmaceutical field, and can be prepared into a composition, according to any techniques well known in the pharmaceutical field, comprising a therapeutically effective amount of said compound in combination with a commonly used carrier or a pharmaceutically acceptable carrier.

The term “carrier” or “pharmaceutically acceptable carrier” used herein includes, but not limited to, pharmaceutically acceptable excipients, fillers, diluents, or the like, including those well known to one of ordinary skills in the pharmaceutical field.

The present invention is explained in the above description of the invention and the following examples, which should not be used to restrict the scope of the present invention.

### EXAMPLE

1. Preparation of the Compound Having Formula I of the Present Invention

Dopamine (1.6 g), 10 mL methanol, 1 mL 1N hydrochloric acid, and 2 mL 99% acetaldehyde were added sequentially into a 50 mL round-bottom flask and stirred for 6 hours under room temperature. The concentrate obtained by reduced-pressure concentration was loaded into a Lobar RP-18 column (size B, Merck), eluted by a 0.05% formic acid aqueous solution, to give ¹H NMR essential pure salsolinol (1.0 g).

Using ESI-TOF mass spectrometry and NMR spectroscopy analysis, the characterization data of salsolinol are as follows:

¹H NMR (CD₃OD, 400 MHz) δ6.63 (1H, s), 6.57 (1H, s), 4.30 (1H, q, J=6.8 Hz, H-1), 3.40 (1H, dt, J=12.6, 5.6 Hz, Hₐ-3), 3.20 (1H, ddd, J=12.6, 8.2, 5.6 Hz, H_{b}-3), 2.92 (1H, ddd, J=16.8, 8.2, 5.8 Hz, Hₐ-4), 2.80 (1H, dt, J=16.8, 5.6 Hz, Hb-4), 1.55 (3H, d, J=6.8 Hz, Me-1); ESIMS: m/z 180 ([M+H]⁺).

2. Evaluation of the Effect of Salsolinol in Treatment of a Wound

Salsolinol ointments at 0.01 mg/g, 0.03 mg/g and 0.1 mg/g were prepared respectively by dissolving 0.5 mg, 1.5 mg, and 5 mg of salsoinol in 2.5 mL of glycerol (Sigma Inc., MO, USA) plus 1.0 mL of Creagel emulsifier (First Chemical, TPE, Taiwan) and 45.5 mL of distilled water. Vehicle was composed of glycerol (2.5 mL), Creagel emulsifier (1.0 mL), and distilled water (45.5 mL).

Guinea pigs were purchased and were acclimatized at a room temperature, and had free access to water. All experimental procedures were approved by and performed in compliance with the guidelines of the Institutional Animal Care and Use Committee (IACUC). All surgeries were conducted while animals were under continuous anesthesia with 4% isoflurane.

The dorsal skin was shaved and then disinfected with 10% povidone-iodine before an excisional full-thickness square-shaped skin wound (1.0×1.0 cm) was induced using a sterile wound maker. Animals were then housed alone in its cage to avoid any further wound damage.

Animals were randomly divided into six groups (n=5 or 6, each group) as follows:
(1) animals treated with salsolinol at a concentration of 0.01 mg/g (C0.01, n=6);
(2) animal treated with the vehicle (V0.01) as compared with the group C0.01;
(3) animal treated with salsolinol at a concentration of 0.03 mg/g (C0.03, n=5);
(4) animal treated with the vehicle (V0.03) as compared with the group C0.03.

The groups were administered at the same amount of the salsolinol at 0.01 mg/g, and 0.03 mg/g or the vechile at the same amount once daily and the wounds were photopictured on Day 1, Day 3, Day 5, Day 7, Day 10, and Day 14. The sizes of the wounds were analyzed using Visual Basic 6.0. The rate of wound recovery at each experimental time point is calculated by the size of the wound as compared with that of the wound at D1, wherein the rate of wound recovery at Day 1 is 1.0.

In this example, the wounds of animals treated with salsolinol were compared with those treated with the vehicle as control groups.

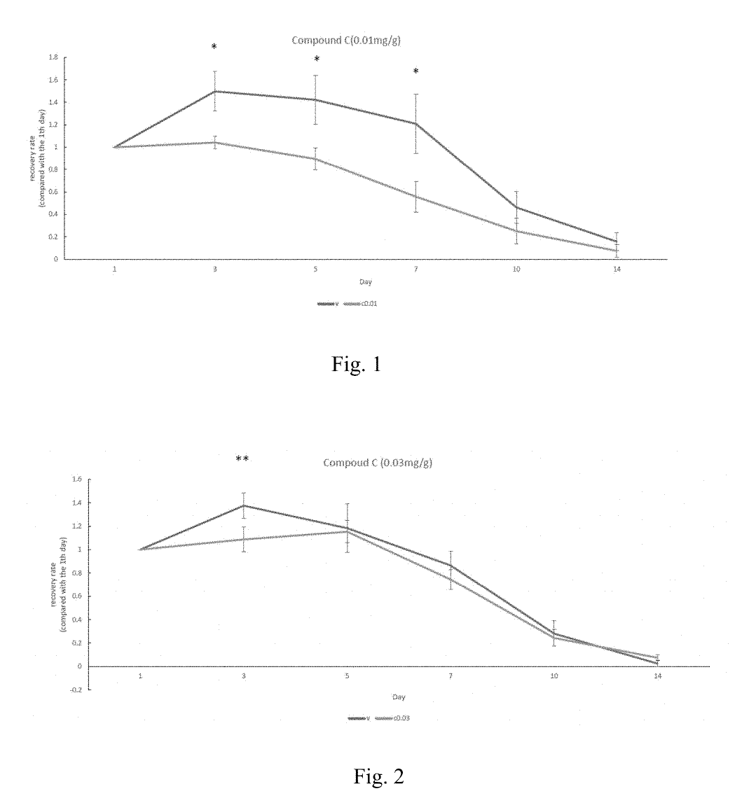

As shown in FIG. 1, the wound healing in the animals treated with salsolinol at a concentration of 0.01 mg/g (C0.01) was significantly better than that of the control group treated with the vehicle (V0.01) after Day 3, Day 5 and Day 7 after the treatment (p<0.01).

As shown in FIG. 2, the wound healing in the animals treated with salsolinol at a concentration of 0.03 mg/g (C0.03) was significantly better than that of the control group treated with the vehicle (V0.03) after Day 3 after the treatment (p<0.05).

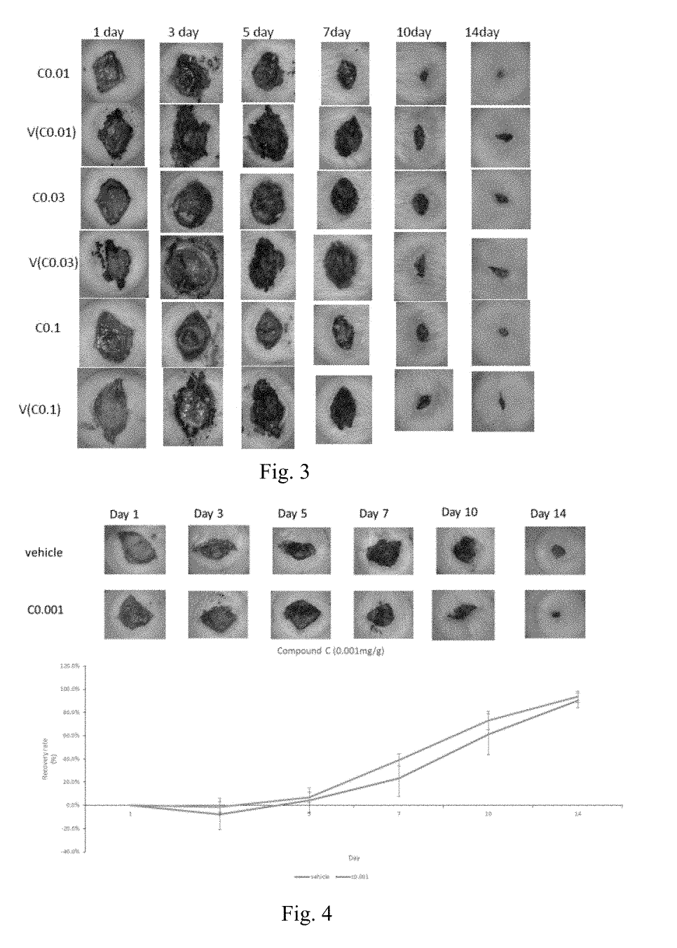

As shown in FIG. 3, the wound recovery in the groups treated with salsolinol at 0.01 mg/g, 0.03 mg/g or 0.1 mg/g was better than the controls treated with the vehicle only.

In another experiment, the wounds of the guinea pigs were treated with salsolinol at a concentration of 0.001 mg/g, 0.01 mg/g or the vehicle at the same amount once daily and the wounds were photopictured on Day 1, Day 3, Day 5, Day 7, Day 10, and Day 14. The sizes of the wounds were analyzed using Visual Basic 6.0. The recovery rate of the wound at each experimental time point was calculated by the size of the wound as compared with that of the wound at D1, wherein the recover rate of wound at Day 1 is 0.0%.

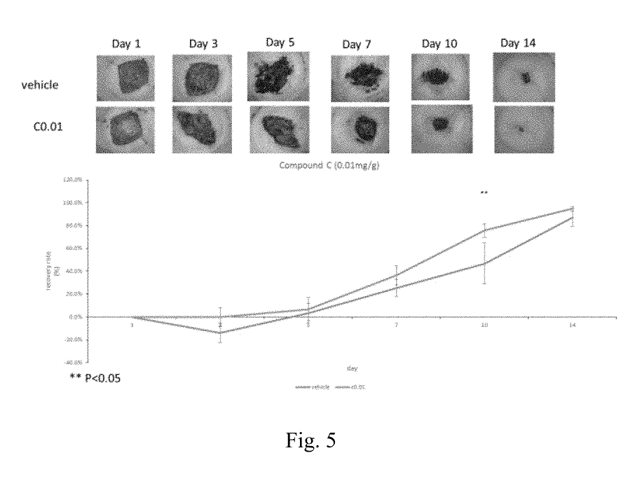

As shown in FIG. 4, the recover rate in the animals treated with salsolinol at a concentration of 0.001 mg/g (C0.001) was better than that of the control group treated with the vehicle. However, as shown in FIG. 5, the recover rate in the animals treated with salsolinol at a concentration of 0.01 mg/g (C0.01) was significantly better than that of the control group treated with the vehicle after Day 3 after the treatment (p<0.05).

In summary, salsolinol at a concentration ranging from 0.001 mg/g to 0.03 mg/g, preferably 0.01 mg/g, enhanced the wound recovery as compared with the control groups (treated with the vehicle). It can be concluded in the present invention that salsolinol at a specific concentration provides a good efficacy in wound healing, and has potential for developing a medicament for wound healing.

I/We claim:

1. A method for wound healing comprising administering to a subject in need thereof a pharmaceutical composition comprising a pharmaceutically acceptable carrier and a therapeutically effective amount of a compound having the general Formula I: 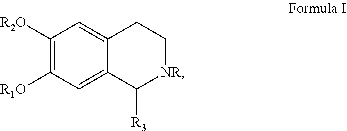
wherein R, R₁ and R₂ are each independently H, alkyl or acyl (RₐCO) group; R₃ is H, alkyl or substituted benzyl group; wherein Rₐ is H or alkyl group.

2. The method of claim 1, wherein said substituted benzyl group has the following Formula II: 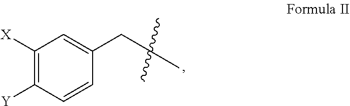
wherein X and Y are each independently H, OH, methoxy (OMe) or acyloxy (R_{b}CO—O—) group;
wherein R_{b} is H or alkyl group.

3. The method of claim 1, wherein the compound is salsolinol.

4. The method of claim 1, wherein the effective amount of the compound is 0.001 mg/g to 0.03 mg/g

5. The method of claim 1, wherein the effective amount of the compound is 0.01 mg/g.
